(19) United States
(12) Patent Application Publication
(10) Pub. No.: US 2015/0297238 A1
(43) Pub. Date: Oct. 22, 2015

(54) GEOMETRIC COIL

(71) Applicant: Christopher G.M. KEN, San Mateo, CA (US)

(72) Inventor: CHRISTOPHER G.M. KEN, SAN MATEO, CA (US)

(21) Appl. No.: 14/646,609

(22) PCT Filed: Aug. 19, 2013
(86) PCT No.: PCT/US2013/055503

§ 371 Date: May 21, 2015

Related U.S. Application Data
(60) Provisional application No. 61/684,777, filed on Aug. 19, 2012.

(51) Int. Cl. A61B 17/12; B21F 3/00

(52) U.S. Cl. CPC A61B 17/12022; B21F 3/00; A61B 2017/00526

(57) ABSTRACT

The invention is an embolic coil having a complex secondary shape based on extensions of the coil into planes orthogonal to the length of the coil. A spring coil is wound on a secondary shaping mandrel having branched extensions positioned sequentially along the length of the linear base mandrel. The spring coil is wound around the base mandrel and the branches that extend or protrude from the base in successive increments creating coil extensions. The secondary wind is stress-relieved. The resulting occupation of space by the secondary shape of the embolic coil is space-filling with extensions into two or more planes, the extensions alternating along the length of the somewhat compressed coil. The invention allows a spring coil to act like a compression coil in its secondary shape.

### CONTINUITY DATA

The application claims priority from an earlier filed provisional application of the same title, filed with the US Patent Office Aug. 19, 2012 and having the Ser. No. 61/684,777.

### TECHNICAL FIELD

The invention relates medical devices comprising implantable embolic coils for treating aneurysms and other vascular defects. The invention further relates to embolic coils having a secondary shape. The invention includes methods of making embolic coils and correcting vascular defects.

### BACKGROUND

Reduction and eventual termination of blood flow to a region of the patient's body experiencing abnormal blood flow is a treatment for certain vascular defects including aneurysms, arteriovenous malformations, traumatic fistulae and tumors. These conditions require that the blood flow through a portion of a blood vessel be stopped, for example by introducing an artificial device into the vessel to slow the flow to allow the natural clotting process to form a more complete blockage. Embolic coils can be inserted into a vascular defect through a catheter, and the coils are detached from the delivery unit once they have been pushed through the catheter and inserted into the vascular abnormality. Inside the defect, the coils encourage the blood to clot.

Embolic coils are usually made from a biocompatible material, to minimize problems associated with tissue irritation and rejection. Coils have been made of platinum, platinum alloy (such as a platinum-tungsten alloy), stainless steel, Nitinol, and Elgiloy® alloy. Typical embolic coils are formed winding a wire over a cylindrical mandrel into a spring, forming what is commonly referred to as a primary coil, and winding the primary coil around another cylindrical mandrel to form a secondary coil. Upon delivery, the helical shape of the secondary coil is enclosed in the vascular defect, twisting into a consolidated mass much like a tangled helical telephone cord. The complex curves fill the space of the vascular defect and slow blood flow into it. In treatment, an embolic coil is inserted in the blood vessel using a catheter, and is placed within the bulging section. Over time, a clot forms around the embolic coil, and blood flow through the weakened section is blocked.

Although the standard of care for aneurysms is coiling, there remains a 30% recanalization rate with coiling for treating aneurysms. Recanalization is often required when the coil begins to slip out of the abnormality, or packs into the space more densely over time so that there remains enough space for abnormal blood flow to resume. Pressure against the vascular wall created by the resumption of abnormal blood flow risks aneurysm rupture and a hemorrhagic stroke. Coil compaction occurs if that the coil loosens from its entanglement, creating more room in the vascular defect. Embolic coils that resist compaction and reduce the current recanalization rate in clinical practice would be a great advantage to the medical community.

### SUMMARY

The invention is a new design of embolic coil. The embolic coil comprises a primary spring coil adapted to assume a secondary shape to fill a vascular defect having an essentially spheroid shape. The secondary shape of the coil comprises protrusions orthogonal to a linear reference, and the protrusions can be spaced along the linear reference at lengths approximating the lengths of the protrusions.

The embolic coil comprises a spring coil stress-relieved (which can be accomplished by heat-treating) on a secondary shaping mandrel. The secondary shaping mandrel about which the spring coil is wound comprises a linear base or a length and a plurality of branches extending or protruding (often sequentially) from the linear base along the length of the shaping mandrel. Each branch extends into and protrudes into and defines a plane orthogonal to the linear base or another protruding member or branch or extension.

The primary spring coil in the secondary wind can be heat-treated to provide stress-relief. Other methods of stress-relief may also be employed. The secondary coil is removed from the shaping mandrel. When the coil reassumes its stress-relieved form, along the length of the secondary coil are extensions of coil protruding or extending in two or more planes, orthogonal either to the base mandrel or each other. Each plane is orthogonal at least to the length of the secondary coil as defined by the linear base of the shaping mandrel.

Several distinct planes can be created from a geometric mandrel, having geometric units from which protrusions extend, along the length of the mandrel. For a secondary coil having two planes, the two planes can be positioned for example 45° apart, 90° apart or 180° apart. Along the length of the coil, the two planes can maintain the same degree of separation from each other, but rotate for example clockwise along the length of the secondary coil. Another configuration can have one plane at 0°, another at 60°, and another at 120° sequentially along the length of the coil. Sequential planes along the length of the coil are at 0°, 60°, and 120°.

Accordingly, the geometry of such a secondary coil can be thought of a triangular, or spherical, or cuboid, or pyramidal. A secondary coil having extensions (also called protrusions) extending or protruding in planes at 0°, 90°, 180°, 270°and 360° (which is the same as the 0° plane) has the geometry of a square or a rectangle. However, note that the geometry is not found in a single cross section of the secondary coil but is drawn out along the length of the coil. The coil is heat-set and thus adapted for constraint in a delivery tube. Upon release of the constrained coil, it is capable of occupying space by assuming the secondary coil form upon release from the tube.

A primary spring coil when wound on such a mandrel and stress relieved will assume a space-filling shape that fills the space of a vascular defect with more material expanded into the defect than a simple primary coil.

The primary spring coil can comprise any material or combination of materials that can form a spring coil. Typically a primary spring coil is formed of a member having a length selected from wire, fiber, strand, cable and filament, or any member capable of being wound on a mandrel to form a spring coil.

The material of the the primary spring coil can be the typical materials that form spring coils, such as a metal, an alloy, a polymer, a combination of a metal and polymer, or a composite of a metal, an alloy or a polymer. Accordingly, the primary spring coil can comprise a material selected from a metal, a metal alloy, a composite, and a polymer, wound into a spring coil.

The embolic coil system is deliverable to vascular defects using a delivery system that can include a delivery member (such as a tube, a catheter, or other delivery tool) for inserting the coil in a vascular defect of a patient, and a delivery release system to release the coil from the tube and place it into the vascular defect.

To make such an embolic coil, a primary coil is wound as a secondary wind along a linear base such as the mandrels described herein. Typically the mandrels for the secondary wind will having alternating sequential extensions that result in protrusions of coil in two or more planes orthogonal to the linear base. The spring coil wound on the mandrels with protrusions is then stress-relieved (which can be accomplished by heat or other methods such as chemicals or electricity) which provides the opportunity for the wound coil to return to that low-energy state upon delivery to a vascular defect. Stress relieving can be accomplished by heat-treating the coil while it is wound along the secondary shaping mandrel.

The stress-relieved coil can be placed in a tube for delivery. Upon release from the tube, the coil assumes the secondary shape in an environment that reverts the coil to its stress-relieved state. The extensions of the secondary coil allow the coil to resist compaction and occupy the space of a vascular defect in a predictable manner. The secondary coil acts as a slight compression coil in that extensions of the primary coil behave with new limitations. When met with a vascular wall, the coil remains soft but carries more dimension than a twisting linear coil. Each protrusion tends to occupy it's own plane, rather than behave as part of the original linear coil. Thus, the coil may be more likely to be retained within the vascular defect. The geometric coil is a controlled and predictable space-filling embolic device.

More specifically, after the coil is formed for treatment of a vascular defect, it is constrained in a delivery tube, and the tube is inserted into a vascular defect, and a coil delivery system pushes the coil out of the tube and into the defect. There the coil assumes its stress-relieved form and occupies the space that is the defect. The invention includes a mandrel having geometric units along the length of the mandrel and protrusions extending from the geometric units. The geometric units can be the same size or they can decrease in size along the length of the mandrel. Such a mandrel can be used to wind the primary coil and form the second wind, that is then stress-relieved to become a geometric coil having the qualities described.

### BRIEF DESCRIPTION OF DRAWINGS

FIG. 1A depicts the planes extending from a cylindrical mandrel. FIG. 1B depicts a branched shaping mandrel having a linear base and branches extending from the base.

FIG. 2A depicts the linear base having planar sections. FIG. 2B depicts a planar sections of the mandrel.

FIG. 3A depicts the shaping mandrel wound with the spring coil. FIG. 3B depicts a secondary wind of the coil.

FIG. 4A depicts the coil in a tubular constraint. FIG. 4B depicts the coil taking its secondary shape as it exits the delivery tube. FIG. 4C depicts the embolic coil being delivered to a vascular defect.

FIG. 5A shows a shaping mandrel having geometric units 3 and also having orthogonal protrusions or extensions 5. FIG. 5B shows mandrel 9 having geometric shapes 7 having protrusions orthogonal to the length of the mandrel 9. FIG. 5C shows a mandrel 11 having decreasing sized geometric protrusions 15 and 13, and also orthogonal protrusions.

### DETAILED DESCRIPTION

The invention is an embolic coil having a controlled pre-designed geometric-like structure and dimension. A primary linear spring coil is wound on a secondary mandrel that is branched for shaping the primary coil. The branches of the mandrel protrude in alternation into the planes orthogonal to a linear plane of the length of the mandrel. After the linear coil is wound along the mandrel and branches the wound coil is stress-relieved (by an appropriate amount of heat for the metal or alloy being used or by some other method such as vibration, chemical treatment, or pH change).

A spring coil or primary coil is made from a wire, strand, cable, or fiber. The spring coil can be made from any material having the length, strength and flexibility to be wound into an expansion spring. For example, the wire of the coil can be a metal, an alloy, a polymer, or a combination of these or other materials. The spring coil is formed by winding the wire on a cylindrical mandrel to form a spring.

The secondary mandrel is generally linear, but it has protrusions orthogonal to the line or length of the mandrel. The mandrel can have a length or linear base such as a rod. The linear base can have, for example, a round, square, triangular or rectangular cross section. The mandrel can also be a rod spaced with geometric units such a spheres or cubes. The protrusions can extend from the geometric units. The geometric units can be the same size, or decreasing size.

The spring coil is wound in a process called secondary shaping. The secondary wind or secondary shaping process is accomplished by winding the spring coil along the secondary shaping mandrel (the rod with extensions, or the rod with geometric units having extensions). The second winding forms a secondary shape for the coil device.

As described, the secondary mandrel has a linear base that is rod-like, or cylindrical, having a cross-sectional geometric shape, and a length. Branches extend from the linear base along its length. The primary coil is wound along the length of the secondary shaping mandrel around the linear base, and successive branches in alternation.

The branches of the shaping mandrel extend approximately perpendicularly from the mandrel along a horizontal plane, each branch occupying a plane orthogonal to a plane of the length-wise mandrel. Effective design of the secondary coil will provide a regular or random balance of extensions from the linear base into planes that are both perpendicular and orthogonal to the linear base, the extensions each occupying their own position along the length of the mandrel so that they have freedom to extend into their perpendicular plane without interference from the other extensions.

The secondary wind is formed as the linear mandrel is wrapped with the primary spring coil generally once, but possibly twice or more, followed by winding the coil around a first branch once, or possibly twice, winding the coil around the base mandrel further down the length of the mandrel, followed by winding the coil on a second branch positioned extending into a second plane perpendicular from the linear base of the mandrel, and so on. The branches can occupy multiple planes, provided they alternate planes with regard to the other branches nearby. This provides a means for the coil extensions of the secondary shape to occupy the plane into which they extend with relative or maximal freedom from interference from other coil extensions.

This patterning also provides balance in the secondary shape, and allows the secondary coil to better occupy space. Effectively, each coil extension is limited in its space-filling ability only by the limits of the constraints of the linear coil, but not by physical interference from nearby coil extensions. Ideally, the coil's secondary shape serves to occupy space in multiple planes extending from the linear base.

The appropriate number of planes that should be occupied in any given coil design has to do, at least in part, with the size of the vascular defect. Larger defects mean that there is more space to fill, and coils having more planes per linear progression of secondary coil will tend to occupy a greater volume. Smaller vascular defects having less space to fill may be best served with implantation of a coil having fewer planes, for example two or three planes extending from the length of the secondary wind, the two or three planes extending in a regular pattern: i.e. the first coil extension occupies plane 1, the second coil extension occupies plane 2, the third coil extension occupies plane 3, the fourth coil extension occupies plane 1 again, the fifth coil extension occupies plane 2, and the sixth coil extension occupies plane 3, and so on.

Thus the branches of the shaping mandrel extend generally perpendicularly from and orthogonal to a plane of the linear base of the mandrel. The branches of a secondary shape occupy a minimum of two planes along the length of the linear base mandrel. Those two or more branches can be in any angular relation to each other (based on a 360° circle), for example, 30° apart, 45° apart, 60° apart, 90° apart, and 180° apart, or a combination of some of these angles of separation. The pattern of branching along the length of the mandrel can be a regular or an irregular pattern. For example, a first branch can extend approximately perpendicularly and orthogonally from a linear mandrel, then at a step further along the linear mandrel, a second branch can extend 180° in relation to the first branch in an extension also perpendicular and orthogonal to the linear mandrel. The degree relationship is defined essentially on a circular but linear (i.e. cylindrical) axis.

Further by example, a second branch can extend a step further along the linear mandrel from the first branch, the second branch extending 90° in relation to the first branch. A third branch can extend from the mandrel a step further along the length of the mandrel, at 90° from the second branch, and 180° from the first branch. A fourth branch can extend from the mandrel a step further along the length of the mandrel, at 90° from the third branch, 180° from the second branch, and 270° (or 90° in a counter direction) from the first branch. Just described is a “square geometric coil”. If, for example, three planes orthogonal to the length of the base mandrel, were defined, each with a branch, the planes could be positioned evenly apart, the first branch 60° from the second branch, the second branch positioned a step further along the linear mandrel from the first branch, the second branch also 60° from the third branch, the third branch positioned yet a step further along the linear base mandrel. A secondary coil so designed would be a “triangular geometric coil” having coil extensions in each of three evenly spaced planes, the planes albeit extending from the linear base coil in regular alternation along a length.

Within the degree of separation established, the branches can rotate along the length of the base mandrel regularly, or randomly. The alternation can occur in steps with successive branches spaced along the linear mandrel a distance of at least about a single wind of the primary coil, or greater. With the branches extending from the linear mandrel having about the spacing of a single wind of the primary coil along the linear branched mandrel, the winding can begin, for example, with a single wind of the primary coil on the base mandrel, followed by a wind on a first branch, followed by a return of the primary coil to a single wind on the base mandrel, followed by a wind on a branched mandrel in a different plane than the preceding (or succeeding) branch, and so on.

Alternation of the branches along the linear base mandrel results in adjacent coils extending into different planes perpendicular and orthogonal to a linear axis of the base mandrel. So derives the concept of the secondary shape as a “geometric coil”. For example, a geometric triangular coil will have a triangular design with three branches alternating into three planes separated by 60° each along the length of the linear base mandrel. A geometric rectangular coil will have 4 branches alternating into 4 planes separated by 45° each along the length of the linear base mandrel, and so on.

The length of the linear base of the secondary shaping mandrel can extend as long as necessary to form an embolic device that, upon deployment, will substantially fill the space within a target vascular defect. Two determinations will generally come into play for estimating how long the secondary coil should be for a given vascular defect. Given a known size, diameter, or volume of a target vascular defect, a first determination is what space a secondary coil will fill given a number of extensions and a number of planes into which the coil extends.

A second determination is then what length of primary spring coil is needed to make the secondary coil of the given number of extensions and the given number of planes. Generally, the more extensions, and the more planes available into which the coil extends will result in a denser but shorter secondary coil. Also, the length of primary coil used for each extension can be calculated, and the number of extensions can be multiplied by the length of primary coil used for each extension to gather a rough estimate of what length of primary coil is needed for a target length of secondary coil.

The greater the number of planes designated for extensions in the design of the secondary coil, the more space-filling the secondary coil will tend to be. Thus, one possible way to estimate what design of secondary coil will best fill a vascular defect of a given size (or how many planes the secondary coil should be designed to extend into) is to multiply a number of planes into which the coil extends in one full design unit and the length of each extension+the overall length that it takes to complete one full design unit along the linear base of the secondary coil. The larger this number, the more space-filling the coil. Larger vascular defects will require an increase in space-filling capacity of an implanted coil.

There may be several ways to figure the design of secondary coil given a known size or shape of vascular defect, including the estimations just described, and also other calculations and considerations. How many branches along the mandrel will determine how many coil extensions the secondary shape has. How many planes into which the coil extends translates into what geometry the secondary coil has, i.e. three planes will form a secondary coil reminiscent of a triangle, four planes will form a secondary coil reminiscent of a square or rectangle, and so on.

In calculating what length of primary coil is needed to generate a secondary coil of a desired length, a formula can be developed that uses the number of extensions in the secondary coil, the number of planes into which there are extensions, and the average length of a “step” from a wind on the base mandrel to a wind on a branch, measured along the length of the linear base of the secondary shaping mandrel. After the primary coil is wound on the secondary shaping mandrel, the coil is heated to provide stress relief so that the coil will retain the secondary shape that has been created by the branched shaping mandrel. After heating to a temperature sufficient to provide stress relief in the material and coil, the coil is removed from the shaping mandrel and can be loaded into a tubular restraint for delivery to a vascular defect in a patient.

The coil is inserted into a vascular defect in a patient's body as a linear coil in a tubular constraint. Upon release from the tubular constraint the coil assumes the secondary “geometric” shape in the vascular defect. The coil design provides the slight effect of a compression coil and is less likely to experience compaction (experienced with the untangling of a tangled coil). The geometric structure of the coil is space filling due to the extensions of coil that extend into planes orthogonal to the length of a central wind (created with the wind of coil along the length of the linear base of the shaping mandrel).

Coils have been typically made of platinum, platinum alloy (such as a platinum-tungsten alloy), stainless steel, Nitinol, and Elgiloy® alloy. The coil can also include one or more polymers, such as polyolefins, polyurethanes, block copolymers, polyethers, and polyimides. A radiopaque material can also be incorporated into the coil, the radiopaque material having a density of about ten grams per cubic centimeter or greater. Typically, the radiopaque material is a metal (e.g., tungsten, tantalum, platinum, palladium, gold, titanium, silver), a metal alloy (e.g., stainless steel, an alloy of tungsten, an alloy of tantalum, an alloy of platinum, an alloy of palladium, an alloy of gold, an alloy of titanium, an alloy of silver), a metal oxide (e.g., titanium dioxide, zirconium oxide, aluminum oxide), bismuth subcarbonate, or barium sulfate. Sometimes the radiopaque material is a contrast agent, such as, for example, Omnipaque™, Renocal™, iodiamide meglumine, diatrizoate meglumine, ipodate calcium, ipodate sodium, iodamide sodium, iothalamate sodium, iopamidol, and metrizamide.

The member forming the primary coil or spring coil can be a single wire (e.g. of metal or metal alloy), a fiber (e.g. of polymer, polymer composite, alloy and polymer, etc.), or a strand, braid or cable. For example, a cable can be made of several filaments of metal, alloy or polymer. For example, 6 filaments of 0.00007″ diameter each can be wrapped around a central wire or filament of the same diameter. The final cable or complex member will be about 0.0002″ and the resulting cable can be used to form the primary spring coil.

The embolic coil is adapted to expand and occupy a spheroid cavity with greater efficiency than the spring coil could prior to winding and heat-treating on the secondary mandrel. Treating a vascular defect comprises inserting the embolic coil into a patient having a vascular defect, and allowing the coil to assume the secondary shape (the stress-relieved shape). The goal is that the embolic coil occupies the defect once it assumes its secondary shape. The secondary shape has an advantage in its space-occupying function because the coil sections extend in directions that help fill the space of the defect.

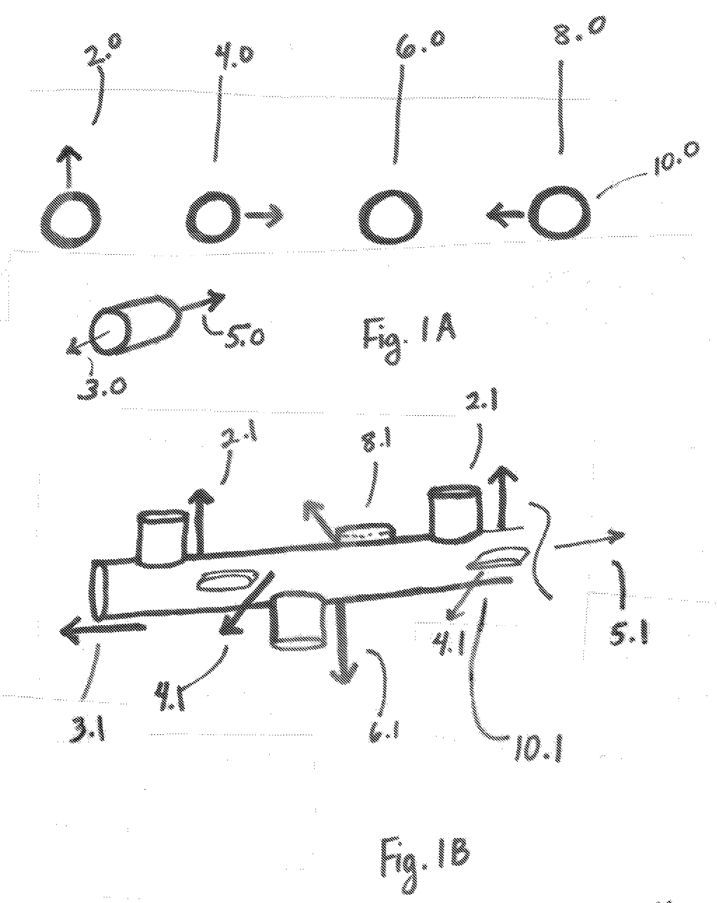

Turning now to the figures, FIG. 1A depicts directions 2.0, 3.0, 4.0, 5.0, 6.0 and 8.0 from cross sections of mandrel 10.0. The directions shown in FIG. 1A indicate the directions for branches 2.1, 3.1, 4.1, 5.1, 6.1, and 8.1 in FIG. 1B positioned along the length of mandrel 10.1. From the 6 directions shown in FIGS. 1A and 1B, the geometric basis of the shaping mandrel 10.1 is a square or cube. The planes such as 2.0 and 4.0 in FIG. 1A become the branches 2.1 and 4.1 respectively in FIG. 1B, and branches 2.1 and 4.1 form the basis of coil extensions in the secondary coil.

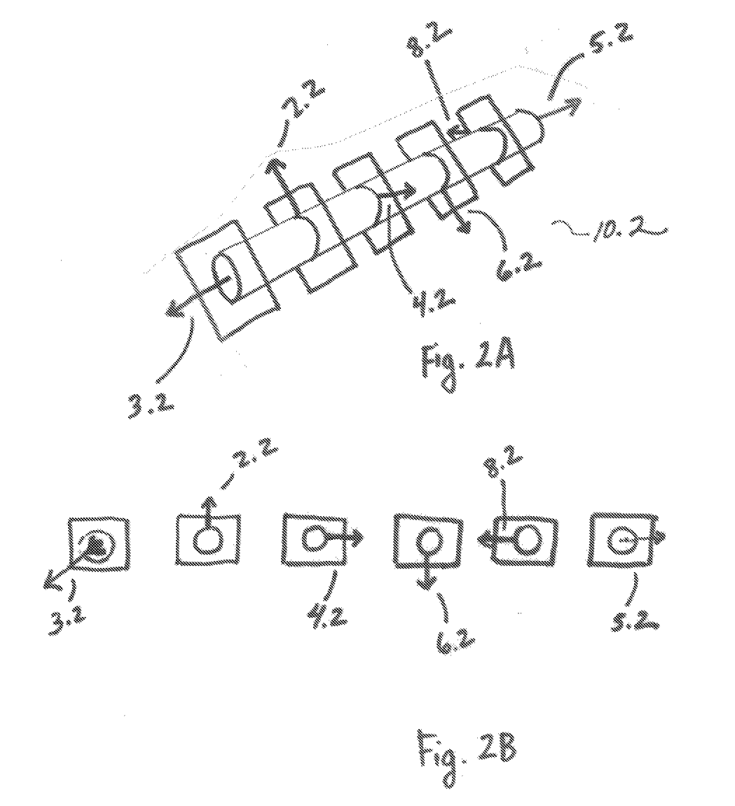

FIG. 2A depicts a secondary shaping mandrel 10.2 having an essentially square or rectangular basis for the shape as indicated by the 4-sided planar surfaces shown as (sequentially along the length of the linear base of mandrel 10.2) 3.2, 2.2, 4.2, 6.2, 8.2, and 5.2. FIG. 2B removes the planes depicted in FIG. 2A to more clearly show the directions into which the coil will extend, i.e. 3.2, 2.2, 4.2, 6.2, 8.2, and 5.2.

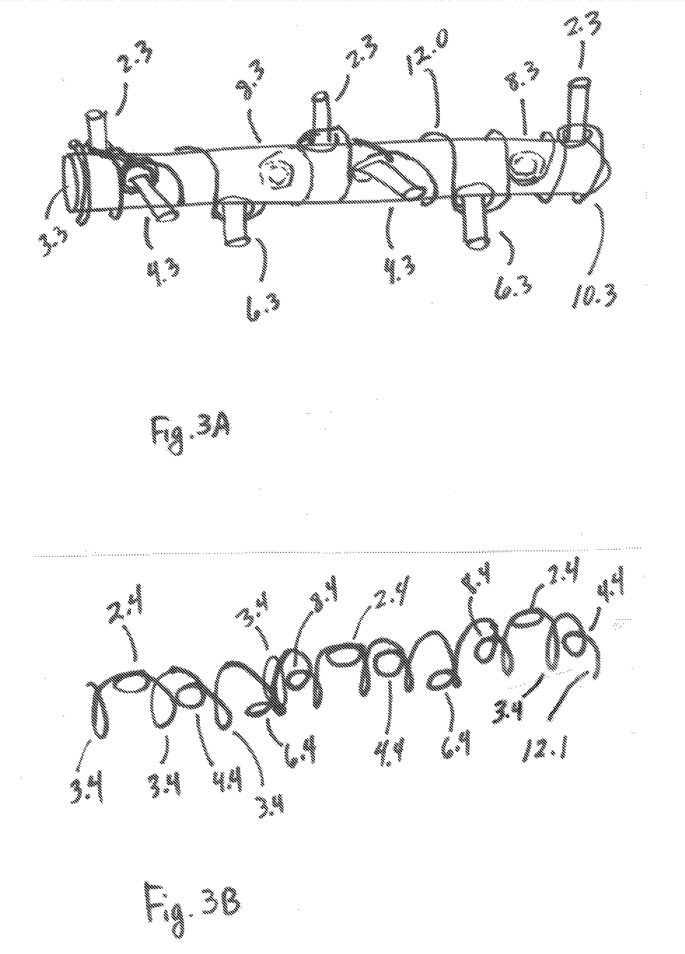

FIG. 3A depicts a fully formed shaping mandrel 10.3 having branches 3.3, 2.3, 4.3, 6.3, and 8.3 that correspond to the directions and planes of FIGS. 1A, 1B, 2A, and 2B. In FIG. 3A, spring coil 12.0 is wrapped along the length of the linear base of secondary shaping mandrel 10.3. Spring coil 12.0 is wound sequentially along the length of mandrel 10.3: a first wind is in the plane of 3.3, a second wind is around branched mandrel 2.3, followed by a wind around 3.3, followed by a wind around branch 4.3, followed by a wind around 3.3, one around 6.3, around 3.3, around 8.3, and starting again with a second branch in the plane 2.3. All the branches are meant to extend essentially orthogonally from base 10.3. All the branches of the shaping mandrel are essentially orthogonal to the length of the mandrel 10.3. FIG. 3B depicts the final coil 12.1 after being heat-set on the shaping mandrel 10.3, and removed. When the coil is allowed to assume secondary shape (depicted in FIG. 3B), coil extensions 3.4, 2.4, 4.4, 5.4, 6.4 extend into the planes previously shown in FIG. 3a.

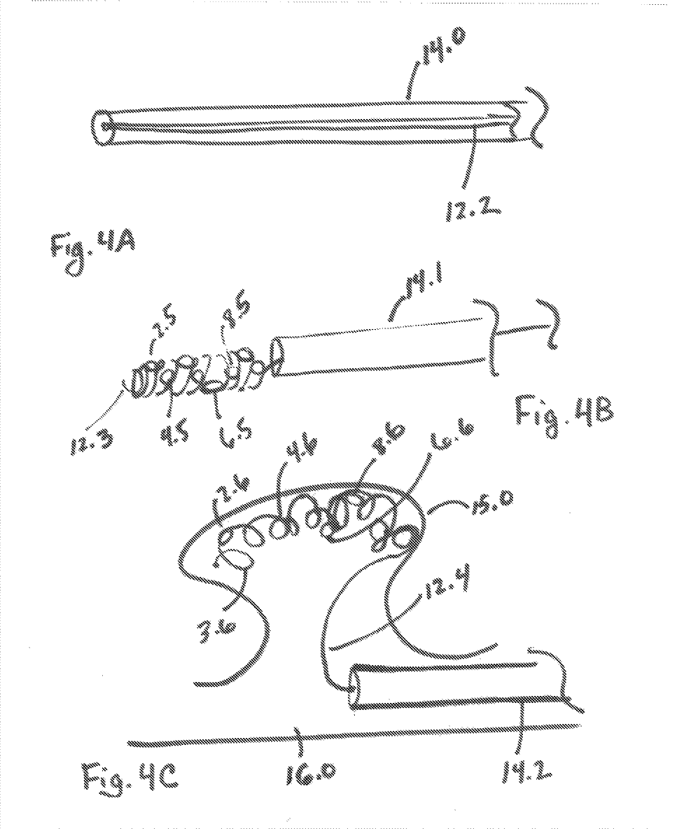

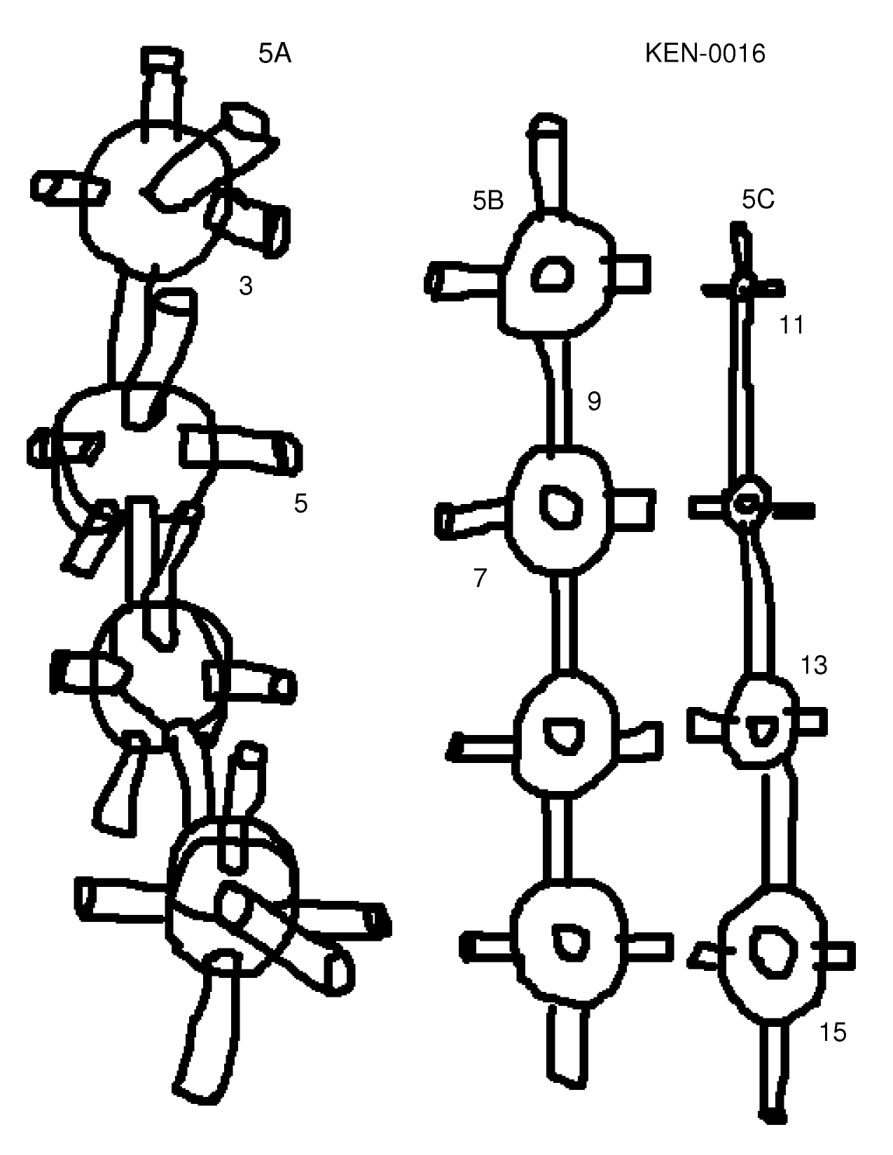

FIG. 4A shows primary coil 12.2 constrained in delivery tube or catheter 14.0 in preparation for delivery to a vascular defect. FIG. 4B shows delivery tube 14.1 releasing embolic coil 12.3 having coil extensions 2.5, 4.5, 6.5 and 8.5. FIG. 4C depicts delivery tube 14.2 positioned in vessel 16.0 at the mouth of vascular defect 15.0 to deliver geometric embolic coil 12.4. Coil 12.4 is linear while constrained in tube 14.2, and assumes its secondary shape when released into vascular defect 15.0, the secondary shape indicated with coil extensions 3.6, 4.6, 6.6, and 8.6.

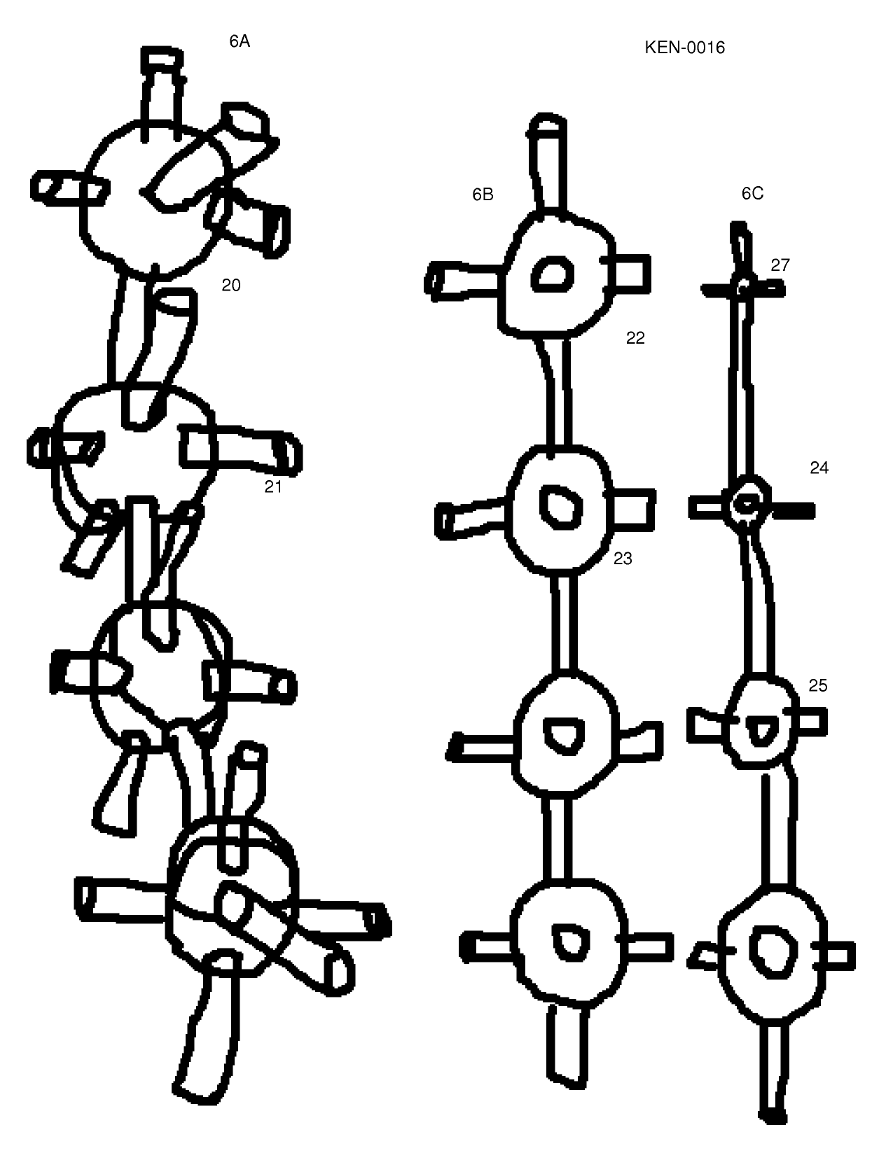

FIGS. 5A, 5B and 5C show mandrels having geometric units 3, 7, 11, and 13. Mandrel 9 had geometric units of the same size, mandrel 15 has geometric units 11 and 13 of decreasing size. Protrusions 5 and 7 extend orthogonally from the geometric shapes.

To make the embolic coil, a length of wire is wound in a primary (linear) coil along a rod-like mandrel. The wire can comprise a single metal, or it can comprise an alloy that is a combination of two or more metals. Other materials such as polymers or fibers can be incorporated into the coil design as benefits the overall implant and its specific purpose. Common alloys for embolic coils can include molybdenum, iron, chromium, nickel, carbon, silicone, manganese, tungsten, phosphorous, sulfur, nitrogen, aluminum, titanium, boron, cobalt, platinum, and any other element found in the periodic table, suitable for making an alloy that can form a wire that can be coiled and also used to create a secondary wind on a branched mandrel, capable also of being stress-relieved in the secondary wind.

After the wire is wound on a linear mandrel, it is removed from the mandrel. The primary coil is then wound a second time on a secondary mandrel and subject to a process that will provide stress relief for the coil. Stress relieving a metal or alloy can be achieved by heating the metal to a suitable temperature, holding the metal at the temperature long enough to reduce residual stresses on the material, then cooling slow enough to minimize the development of new residual stresses. The appropriate temperature for relieving stress in a given alloy will depend on the composition of the alloy. Heat treating metal or alloy is a way to temper or stress relieve the metal or alloy. Later, the metal or alloy will generally choose to assume the shape having less stress.

The size of the primary coil and secondary wind is based on the size of the target vascular defect and the diameter of the vessel in which the coil is delivered. Typical vessel sizes are about 2 mm in diameter (or 0.08 inches). Vascular defects, including cerebral aneurysms, range in size from less than 5 mm in diameter to larger than 25 mm. The primary wind is delivered in a tube that can navigate a 2 mm (0.08″) diameter vessel, taking into account the diameter of such a tube (0.05″ OD; 0.04″ ID). The primary coil ID is likely no more than about 0.0018″, and more likely no more than about 0.0015″. The secondary shape will be based on the length of the primary coil, and the choice of geometry (i.e. three-sided, four-sided, five-sided polygon) and can be designed for various target volumes ranging from a spheroid having about 5 mm diameter to as much as a spheroid having about 25 mm diameter or more. The length of the primary coil will shrink substantially with the secondary wind.

The coil, delivered linearly in a tubular constraint, assumes the lower energy configuration of the secondary wind, and the coil protrusions operate more or less individually to occupy space in a vascular defect defined by the plane of the branch on which the protrusion was formed. The polygonal basis of the geometric dimension of the coil can be regular having protrusions of approximately the same length and spacing along the base mandrel, such as triangular, square, rectangular, pentagonal, hexagonal, etc., or the coil protrusions can be semi regular, based on a regular polygon, but randomly distributed along the length of the base mandrel. Finally, the protrusions can be irregular and random with protrusions of different lengths extending at random in open positions along a line defined by the base of the secondary shape-forming mandrel.

The secondary coil design may provide better coil retention in the vascular defect by exhibiting behavior of a slight compression spring. Each coil subunit or protrusion is allowed expansion in its open plane resulting in a coil having a three-dimensional geometry less likely to be subject to unwinding or the phenomenon known as “compaction.”

It is contemplated that any optional feature of the inventive variations described may be set forth and claimed independently, or in combination with any one or more of the features described herein.

Reference to a singular item, includes the possibility that there is a plurality of the same items present. More specifically, as used herein and in the appended claims, the singular forms “a” and “the” include plural referents unless specifically stated otherwise. In other words, use of the articles allow for at least one of the element in the description above as well as the claims below. It is further noted that the claims may be drafted to exclude any optional element. As such, this statement is intended to serve as antecedent basis for use of such exclusive terminology as “solely”, “only” and the like in connection with the recitation of claim elements, or use of a negative limitation.

The breadth of the present invention is not to be limited to the examples provided and/or the subject specification, but rather only by the scope of the claim language. All references cited are incorporated by reference in their entirety. Although the foregoing invention has been described in detail for purposes of clarity of understanding, it is contemplated that certain modifications may be practiced within the scope of the appended claims. The earlier filed provisional application U.S. Ser. No. 61/684,777 to which this application claims priority is herein incorporated by reference in its entirety.

We claim:

1. An embolic coil comprising:
a spring coil wound and stress-relieved on a secondary shaping mandrel, said secondary mandrel comprising a plurality of branches extending along the mandrel, each branch extending into and defining a plane orthogonal to the mandrel, wherein
the embolic coil is adapted to expand and occupy an spheroid cavity with greater efficiency than the spring coil prior to winding and heat-treating on the secondary mandrel.

2. The embolic coil of claim 1, wherein stress-relieved comprises heat-treated.

3. The embolic coil of claim 1, adaptable for constraint in a delivery tube.

4. The embolic coil of claim 1, wherein the primary spring coil comprises a material selected from wire, fiber, strand, cable and filament.

5. The embolic coil of claim 1, wherein the primary spring coil comprises a material selected from metal, metal alloy, composite, and polymer.

6. The embolic coil of claim 1, comprising a spring coil wound on a linear secondary shaping mandrel having geometric units extending along the mandrel.

7. The embolic coil of claim 5, said geometric units comprising about equal size.

8. The embolic coil of claim 5, said geometric units comprising decreasing size.

9. An embolic coil comprising a primary spring coil adapted to assume a secondary shape to fill a vascular defect having a spheroid shape, the secondary shape of the coil comprising protrusions orthogonal to a linear reference, said
protrusions spaced along said linear reference at lengths approximating the lengths of the protrusions.

10. The embolic coil of claim 9, comprising a spring coil wound on a mandrel having equal geometric units.

11. The embolic coil of claim 9, comprising a spring coil wound on a mandrel having geometric units of decreasing size along the length of the mandrel.

12. A method of making an embolic coil for insertion in a vascular defect of a patient, the method comprising:
forming a secondary wind of a primary spring coil on a shaping mandrel, the shaping mandrel having a length and alternating sequential branches in two or more planes orthogonal to and along the length, and
stress-relieving the secondary wind.

13. The method of claim 12, wherein stress-relieving comprises heat-treating.

14. The method of claim 12, further comprising:
removing the stress-relieved secondary wind from the shaping mandrel, and
constraining the coil in a delivery tube for insertion in the vascular defect.

15. A method of treating a vascular defect, comprising:
loading into a tubular constraint an embolic coil of claim 9 comprising a spring coil having a stress-relieved secondary shape, the secondary shape comprising orthogonal extensions along a length of the spring coil,
inserting the embolic coil into a patient having a vascular defect, and
allowing the coil to assume the secondary shape, wherein an embolic coil occupies the defect in the secondary shape.

16. A secondary shaping mandrel for shaping the coil of claim 9, said mandrel comprising a linear member, said linear member comprising protrusions on which to wind the spring coil, said protrusions protruding orthogonally from said linear member.

17. The mandrel of claim 16, further comprising geometric shaping units spaced along the linear member.

18. The mandrel of claim 17, comprising protrusions orthogonal to an axis of the geometric shaping units.
